(19) United States
(12) Patent Application Publication
(10) Pub. No.: US 2024/0315987 A1
(43) Pub. Date: Sep. 26, 2024

(54) ORAL COMPOSITIONS COMPRISING KETAMINE COMBINED WITH SUBCUTANEAL OR INTRAVENOUS KETAMINE FOR USE IN THE TREATMENT, CONTROL OR PREVENTION OF DEPRESSIVE DISORDERS

(71) Applicant: NEUROCENTRX PHARNA LTD., Edinburgh (GB)

(72) Inventors: Carmel REILLY, Edinburgh (GB); Mario Francisco Pereira JURUENA, London (GB)

(21) Appl. No.: 18/257,384

(22) PCT Filed: Dec. 17, 2021
(86) PCT No.: PCT/GB2021/053345

§ 371 Date: Jun. 14, 2023

(30) Foreign Application Priority Data
Dec. 17, 2020 (GB) 2019952.7

(51) Int. Cl. A61K 31/135; A61K 9/00; A61K 9/48; A61K 45/06

(52) U.S. Cl. CPC A61K 31/135; A61K 9/0019; A61K 9/4858; A61K 9/4866; A61K 45/06

(57) ABSTRACT

The disclosure provides novel compositions comprising ketamine and/or pharmaceutically acceptable salts, enantiomers, derivatives or metabolites thereof as an active pharmaceutical agent for use in the treatment, control or prevention of depressive disorders, including, for example treatment resistant depression (TRD). The disclosure further provides method and uses of combined doses of ketamine showing exceptional efficacy in the treatment of depressive disorders (including TRD) and in the control and duration of remission from the same.

### FIELD

The disclosure relates to novel compositions comprising ketamine. The compositions may have suitable properties that facilitate their manufacture (e.g. at scale and/or in an automated process). The compositions may provide a short term (or rapid or immediate) release of ketamine and/or allow the stable storage of ketamine for extended periods of time. The compositions may be used in the treatment, prevention, management and/or control of a variety of clinical indications including pain, neurological disorders, psychological disorders, mood disorders such as depressive disorders and/or suicidal ideation and behaviour. The disclosure further relates to the treatment of depressive disorders using a combination of orally administered ketamine together with a second anti-depressant agent or after treatment with other anti-depressants.

### BACKGROUND

Over 300 million worldwide people suffer from depression. Existing medications often take many weeks to work, or to fail. Of those treated, between 30 and 50% fail to respond to medication. Patients who fail to respond to different medications are sometimes termed treatment resistant depression (TRD) patients (typically after two successive failures to respond to different medications). About 17% of TRD patients may attempt suicide. (WHO data).

Ketamine is on the World Health Organisation's list of essential medicines for human health. Although it is best known medically as an anaesthetic for human and animal health, and is “approved” or “licensed” by regulatory authorities for this purpose, it is also used worldwide for pain relief, although not licensed for this use. Despite being discovered in the 1960's, Ketamine's potential to treat a range of human diseases and disorders is only recently becoming understood.

More recently, clinical trials have shown that some patients with depression can have a rapid response to ketamine. In particular, studies have shown that injectable intravenous (IV) ketamine elicits a rapid antidepressant response in Major Depression Disorder (MDD) and Treatment-resistant depression (TRD) (Krystal J H, Abdallah C G, Sanacora G, Charney D S, Duman R S. Ketamine: A Paradigm shift for depression research and treatment. Neuron 2019; 101:774-8). It is reported to act within 24 hours and lasts for 4-7 days (transient effect) after a single IV administration of subanaesthetic doses (0.5 mg/kg) (Gautam, CS & Mahajan, Sonia & Sharma, Jatin & Singh, Harmanjit & Singh, Jagjit. (2019). Repurposing Potential of Ketamine: Opportunities and Challenges. Indian Journal of Psychological Medicine. 42. 10.4103/IJPSYM.IJPSYM_228_19). However, the use of an injectable form of an active agent often requires costly hospital stays, or appointments, and inconvenience for the patient.

A nasal spray comprising S-Ketamine (Spravato®) has been developed by J&J (Janssen) and was approved for Treatment Resistant Depression (TRD) in 2019 by the EMA and FDA and is the first licensed ketamine treatment for Depression. However, a number of disadvantages have been reported including a lack of patient compliance, inconsistency of delivery and an unpleasant taste, some patients also report side effects including nausea and vomiting. In addition, the nasal spray must be administered under supervision in a hospital, clinic or surgery. Appropriate spray containers are also more costly to manufacture and less environmentally friendly. In a separate clinical pilot study using a different ketamine nasal spray it was reported that dose-escalation caused issues including high blood pressure, psychotic-like effects and motor incoordination which left some participants unable to continue to self-administer the nasal spray. The pilot study was eventually suspended after testing with five participants due to unexpected problems with tolerability (https://sciencebeta.com/ketamine-spray-side-effects/)

Orally administered ketamine may offer many advantages to the patient. In particular, it offers the potential for a fast-acting and effective treatment for patients. In addition, an oral formulation of ketamine may provide a low cost treatment option with increased patient compliance/acceptability and/or potential for at-home use.

In 2019, a systematic review of oral ketamine in depression concluded from studies that oral ketamine has significant antidepressant effects with good overall tolerability, with antidepressant effects less rapid in onset compared to intravenous ketamine (Rosenblat J D, Carvalho A F, Li M, Lee Y, Subramanieapillai M, Mcintyre R S. Oral Ketamine for Depression: A Systematic Review. J Clin Psychiatry. 2019; 80(3): 18r12475. Published 2019 Apr. 16. doi: 10.4088/JCP.18r12475). These studies mainly used small pharmacy stock prepared oral liquid ketamine (an unlicensed special) or sublingual ketamine (an unlicensed special) as at this time no licensed commercial supply of ketamine exists worldwide, apart from intravenous ketamine licensed for anaesthesia and Nasal Es-ketamine spray (Janssen) licensed for depression based indications.

It has been noted by others that the bioavailability and pharmacokinetics of oral ketamine has been poorly studied (see, for example, “Oral ketamine for depression, 1: Pharmacologic Considerations and Clinical Evidence” (Andrade C. J Clin. Psychiatry. 2019; 80(2): 19f12838); and “Oral ketamine for depression. 2: practical considerations” (Andrade. C. J. Clin. Psychiatry, 2019; 80(2) 19f12838)

A number of oral ketamine formulations are described in WO 2019/243791. The formulations described in that document are in an encapsulated form and exhibit abuse deterrent properties. These formulations have been shown to provide a rapid release of ketamine. However, in some instances, the inventors observed that, in the presence of certain excipients, the ketamine was prone to some level of degradation.

The disclosure provides further ketamine compositions (suitable for oral administration) that may be produced cost-effectively and/or scale, exhibit a desired dissolution profile (for a rapid release of ketamine) and/or which may show an improved stability for extended periods of time. Additionally, the disclosure further provides treatments for a depressive disorder based on the use of an orally administered ketamine in combination with a second anti-depressant agent.

### Ketamine Background

Ketamine is absorbable by intravenous, intramuscular, oral, and topical routes due to both its water and lipid solubilities. When administered orally, it undergoes first-pass metabolism, where it is transformed in the liver by CYP3A4 (major), CYP2B6 (minor), and CYP2C9 (minor) isoenzymes into norketamine (through N-demethylation) and finally dehydronorketamine. Intermediate in the biotransformation of norketamine into dehydronorketamine is the hydroxylation of norketamine into hydroxynorketamine by CYP2B6 and CYP2A6. Dehydronorketamine, followed by norketamine, is the most prevalent metabolite detected in urine. As the major metabolite of ketamine, norketamine is less potent as an anaesthetic, and plasma levels of this metabolite are three times higher than ketamine following oral administration.

Bioavailability through the oral route according to limited studies discussed above reaches approximately 17-20%; bioavailability through other routes are: 93% intramuscularly, 8-50% intranasally, up to 30% sublingually, and up to 30% rectally. Peak plasma concentrations are reached within a minute intravenously, 5 to 15 minutes intramuscularly, and 30 minutes orally. Ketamine's duration of action in a clinical setting is 30 minutes to 2 hours intramuscularly and 4 to 6 hours orally.

The metabolites of ketamine are believed to be important. Taken orally, ketamine that reaches the stomach is then subject to first-pass metabolism and largely converted to norketamine. Norketamine is postulated to provide pain relief and have similar clinical benefits to ketamine with reduced side-effects. However, it is not known if norketamine is an effective clinical treatment at this time. Other metabolites are postulated to be important, particularly those metabolites that are derived from R-ketamine, namely 6-hydroxynorketamine or R-hyroxynorketamine (HNK) and specifically (2S,6S; 2R,6R)-HNK, is responsible for antidepressant-like effects of ketamine in mice. Specifically, administration of (2R,6R)-HNK demonstrated ketamine-type antidepressant-like effects, and preventing the metabolic conversion of ketamine into HNK blocked the antidepressant-like effects of the parent compound.

### Optical Isomers of Ketamine:

Ketamine exists in different optical isomers and salt forms. Some of these forms and metabolites have been shown to have positive clinical utility, with increased or decreased side-effects.

The optical rotation of a given enantiomer of ketamine can vary between its salts and free base form. The free base form of (S) ketamine exhibits dextrorotation and is therefore labelled (S) (+) ketamine. However, its hydrochloride salt shows levorotation and is thus labelled (S) (−) ketamine hydrochloride. The difference originates from the conformation of the cyclohexanone ring. However, not all salts of ketamine show different optical rotation to the free base: (S)-ketamine (R,R)-tartrate is levorotatory, like (S) ketamine.

### Ketamine Mechanism:

The full mechanism, pathways and combination of pathway effects required for ketamine to be effective in different clinical areas is not currently understood. Ketamine is known mostly as an NMDA antagonist, and more recently as an regulator of AMPAR (α-amino-3-hydroxy-5-methyl-4-isoxazolepropionic acid receptor and also referred to as AMPA receptor). However ketamine also has effects at the opioid, sigma, dopamine, serotonin, muscarinic, nicotinic, and oestrogen receptors and inhibits cholinesterase, serotonin, norepinephrine and dopamine reuptake inhibitor, PCP site 2 ligand, blocks sodium and calcium channels, HCN1 cation channels and inhibits nitric oxide synthase.

With a few exceptions (including interactions with the D2high receptor, nicotinic acetylcholine receptors (by metabolites), ERα, and HCN1 channels), these actions are far weaker than ketamine's antagonism of the NMDA receptor, though it has been postulated that allosteric binding at other sites may augment ketamine's activity at these sites.

Metabolites of ketamine including dehydronorketamine, hydroxynorketamine, and norketamine have been found to act as negative allosteric modulators of the α7 nicotinic acetylcholine receptor in the KXa7R1 cell line (HEK293 cells transfected with rat nicotinic acetylcholine receptor genes) with subanaesthetic and nanomolar potencies (e.g., IC50=55 nM for dehydronorketamine), whereas ketamine itself was inactive at the same concentrations (<1 μM). These findings suggest that metabolites may contribute importantly to the pharmacodynamics of ketamine by means other than NMDA receptor antagonism.

It has yet to be fully understood how ketamine mediates its robust and rapid-onset antidepressant effects. In any case, it has been elucidated that acute blockade of NMDA receptors in the brain results in an activation of α-amino-3-hydroxy-5-methyl-4-isoxazolepropionic acid receptors (AMPA receptors), which in turn modulate a variety of downstream signalling pathways to influence neurotransmission in the limbic system and mediate antidepressant effects of NMDA receptor antagonists like ketamine. Such downstream actions of this activation of AMPA receptors include upregulation of brain-derived neurotrophic factor (BDNF) and activation of its signalling receptor tropomyosin receptor kinase B (TrkB), activation of the mammalian target of rapamycin (mTOR) pathway, deactivation of glycogen synthase kinase 3 (GSK-3), and inhibition of the phosphorylation of the eukaryotic elongation factor 2 (eEF2) kinase. In addition to blockade of the NMDA receptor, an active metabolite of ketamine known as hydroxynorketamine, which does not interact importantly with the NMDA receptor but nonetheless indirectly activates AMPA receptors similarly, may also or alternatively be involved in the rapid-onset antidepressant effects of ketamine. Recent research has elucidated that an acute inhibition or sort of “reset” of the lateral habenula, a part of the brain in the limbic system that has been referred to as the “anti-reward centre” (projecting to and inhibiting the mesolimbic reward pathway and modulating other limbic areas), may be responsible for the antidepressant effects of ketamine.

### SUMMARY

The disclosure provides novel compositions comprising ketamine and/or pharmaceutically acceptable salts, enantiomers, derivatives or metabolites thereof as an active pharmaceutical agent. The compositions show good flowability and blending properties which facilitate manufacture. In particular, the disclosed compositions may be particularly suitable for filling capsules to provide encapsulated compositions e.g. in an automated capsule filling process. It has further been identified that ketamine shows good stability in the described compositions with minimal degradation of the active pharmaceutical agent. Consequently, the compositions may allow for the long-term storage of this active pharmaceutical agent. In addition to these favourable stability properties, the compositions may show a desirable release profile, allowing for a controlled delivery of this active pharmaceutical agent. For example, the compositions may provide a rapid and/or immediate release of ketamine.

The disclosure further provides ketamine for use in combination with a second therapeutic agent. For example, ketamine may be administered orally in combination with a second anti-depressant agent to supplement and/or complement a new or existing course of anti-depressant treatment. The oral administration of ketamine can find use in the control, treatment, prevention and/or management of period of instability in depression levels, a relapse in depression levels, an acute depressive episode and/or suicidality/suicidal ideation or behaviour associated with such.

According to a first aspect of the disclosure there is provided a composition comprising an active agent, wherein the active agent is selected from ketamine and pharmaceutically acceptable salts, enantiomers, derivatives or metabolites thereof. The composition may further comprise one or more excipients.

The compositions described herein may comprise any form of ketamine which is able to elicit a desired therapeutic effect (e.g. any form of ketamine which elicits an anti-depressant effect in a subject in need thereof). By way of further example, the disclosed compositions may comprise any form of ketamine which is a NMDA antagonist and/or modulator of the AMPAR (α-amino-3-hydroxy-5-methyl-4-isoxazolepropionic acid receptor).

Ketamine (C₁₃H₁₆ClNO) is a water-soluble phencyclidine derivative with an asymmetric carbon atom; the structure of which is illustrated below.

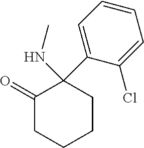 [SMILES: CNC1(c2ccccc2Cl)CCCCC1=O]

Ketamine has two enantiomers: S-(+)-ketamine (sometimes referred to as esketamine) and R-(−)-ketamine (also referred to as arketamine). By way of example, the compositions described herein may comprise one or both of the R-/S-ketamine enantiomers. For example, the composition may comprise either R-ketamine or S-ketamine. Alternatively, the composition may comprise both of the R- and S-enantiomers, optionally as a racemic mixture. Ketamine may also exist as the variant Methoxetamine (https://pubchem.ncbi.nlm.nih.gov/compound/Methoxetamine)

The compositions may comprise a pharmaceutically acceptable salt of ketamine. In some cases, the pharmaceutically acceptable salt may be formed from a racemic mixture of ketamine. In other cases, the pharmaceutically acceptable salt may be formed from a single enantiomer of ketamine (e.g. either R-ketamine or S-ketamine).

Suitable pharmaceutically acceptable salts include acid addition salts formed with organic carboxylic acids such as acetic, lactic, tartaric, maleic, citric, pyruvic, oxalic, fumaric, oxaloacetic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids. In some instances, suitable pharmaceutically acceptable salts may be selected from hydrochloride and tartrate.

The compositions may comprise ketamine hydrochloride. In some cases, the compositions comprise racemic (R,S) ketamine hydrochloride. In other instances, the compositions may comprise (S)-(−)-ketamine hydrochloride or (R)-(+)-ketamine hydrochloride. Ketamine, its metabolites, salts and variants can also exist in deuterated hydrogen forms.

The compositions described herein (which may be formulated for oral administration) may be metabolised by the body to one or more ketamine metabolites. Ketamine is metabolized in the liver via N-demethylation and ring hydroxylation pathways and the main metabolites are norketamine and its hydroxylated derivatives. Consequently, ketamine metabolites may include norketamine and hydroxynorketamine. Other ketamine metabolites include dehydroxyketamine and dehydronorketamine.

By way of further example, the term “ketamine metabolites” may embrace the following compounds:
R-Norketamine;
R-Hydroxynorketamine;
R-Dehydroxyketamine;
R-Dehydronorketamine;
S-Norketamine;
S-Hydroxynorketamine;
S-Dehydroxyketamine;
S-Dehydronorketamine;
2R,6R-Hydroxynorketamine hydrochloride (this enhances AMPA currents; decreases D-serine (a NMDA co-agonist) and lacks ketamine-related side effects).

2S,6S-Hydroxynorketamine hydrochloride (this decreases D-serine (a NMDA co-agonist); it is an antidepressant).

cis-6-Hydroxynorketamine hydrochloride (this enhances AMPA currents: it is an antidepressant)

(S)-(+)-Ketamine hydrochloride (a NMDA receptor antagonist; enantiomer of ketamine hydrochloride with neuroprotective effects).

Norketamine hydrochloride (a potent, non-competitive NMDA antagonist; antinociceptive)
(R)-Norketamine hydrochloride (a NMDA receptor modulator and an analgesic)
(S)-Norketamine hydrochloride (a NMDA receptor modulator and an analgesic).

It should be noted, that the term “ketamine”, “active”, “active agent”, or “active pharmaceutical ingredient” as used hereinafter, shall be taken to refer to and/or embrace all of the ketamine types, salts, derivatives, enantiomers and/or metabolites described above.

The composition may comprise any suitable amount of ketamine. For example, the composition may comprise between about 1 wt % and 50 wt % of the active agent by total weight of the composition. By way of further example, the composition may comprise between about 5 wt % and 30 wt %, or between about 10 wt % and 25 wt % of the active agent by total weight of the composition. For example, the composition may comprise between about 10 wt % and 15 wt %, or between 11 wt % and 12 wt % by total weight of the composition. In other examples, the composition may comprise between about 20 wt % and 25 wt % of the active agent, or between about 22 wt % and 24 wt % of the active agent by total weight of the composition. By way of further example, the composition may comprise about 9 wt %, 10 wt %, 11 wt %, 11.5 wt %, 12 wt %, or 13 wt % of the active agent by total weight of the composition. In other examples, the composition may comprise about 20 wt %, 21 wt %, 22 wt %, 23 wt %, 24 wt %, or 25 wt % of the active agent by total weight of the composition

For example, a composition of this disclosure may comprise between about 5 wt % and 30 wt % of ketamine hydrochloride. In one example, the composition may comprise about 11.5 wt % (e.g. +/−1, 2, 3, 4 or 5%) of ketamine hydrochloride by total weight of the composition. In a further example, the composition may comprise about 23 wt % (e.g. +/−1, 2, 3, 4 or 5%) of ketamine hydrochloride by total weight of the composition

The composition further comprises one or more excipients.

The excipients for use in the compositions may be selected on the basis of function. For example the compositions described herein may comprise excipients which are:
(i) diluents;
(ii) carriers;
(iii) solvents;
(iv) plasticisers;
(v) surfactants;
(vi) lubricants;
(vii) dispersants;
(viii) viscosity modifiers;
(ix) bioavailability modifiers;
(x) emulsifiers;
(xi) penetration enhancers;
(xii) release modifiers;
(xiii) gelling agents;
(xiv) stabilisers;
(xv) antioxidants;
(xvi) compression aids;
(xvii) pH regulators/acidity modifiers; and
(xviii) thermal stability/setting regulators.

For example, a composition as disclosed herein may comprise one or more excipients selected from the group consisting of diluents, lubricants and compression aids.

The precise amount (for example percentage by weight) of each excipient may vary depending on the desired properties of the composition. In particular, the amount and type of excipient may be selected in order to provide one or more (or all) of the following:
(i) suitable blending properties;
(ii) suitable flowability;
(iii) good stability of the active pharmaceutical ingredient (e.g. ketamine or a pharmaceutically acceptable salt form thereof); and
(iv) desirable release properties of the active pharmaceutical ingredient (e.g. ketamine or a pharmaceutically acceptable salt form thereof).

In some cases, a composition with suitable flowability properties can facilitate manufacture (e.g. these properties may facilitate a filling step in an automated capsule filling process).

A compressibility index (C) provides an indication of the flowability of a composition (e.g. a powder composition). A smaller value indicates more favourable flowability properties. The compressibility index may be determined with the formula below.

$$C = 100 ( 1 - {ρ}_{B} / {ρ}_{T} )$$

Where ρ_{B} is the freely settled bulk density of the composition and ρ_{T} is the tapped bulk density of the composition after tapping down.

In some examples, the compressibility index of the composition may be less than 30, 29, 28, 27 or 26. In some instances, the compressibility index may be 25 or less. Thus, appropriate types and amounts of excipients may be selected to provide a compressibility index of 25 or less.

The excipients may be present in any amount between about 1 wt % and 99 wt % by total weight of the composition. For example, the composition may comprise between about 50 wt % to 95 wt %, or between about 75 wt % and 90 wt % of excipients by total weight of the composition.

The excipients may include sugars such as monosaccharides and disaccharides. By way of example, the excipients may include lactose or lactose derivatives. Various different forms of lactose may be used in the compositions described herein. In the compositions described herein, lactose monohydrate may typically be used. However, in some instances, the compositions may alternatively or additionally comprise anhydrous lactose or spray dried lactose. By way of further example, the lactose may be a spray-dried monohydrate lactose.

The excipients may include stearic acid and/or a metal salt (e.g. for use as a lubricant). Suitable metal salts include metal stearates, such as magnesium or sodium stearate.

The excipients may include natural polymers, such as cellulose (such as microcrystalline cellulose), and derivatives thereof (such as ethyl cellulose, (hydroxypropyl)methyl cellulose (HPMC) and hydroxypropyl cellulose (HPC)). In some examples, the composition comprises microcrystalline cellulose (MCC), such as Avicel® PH101 obtained from DuPont.

Suitable excipients for use in the compositions described herein include, but are not limited to, lactose, such as lactose monohydrate, stearic acid and salts thereof, such as magnesium stearate, and microcrystalline cellulose.

By way of further example, the compositions may comprise the following excipients:
(i) lactose, such as lactose monohydrate;
(ii) metal stearate, such as magnesium stearate; and
(iii) optionally, cellulose or a cellulose derivative, such as microcrystalline cellulose.

A lactose excipient (such as lactose monohydrate), or a suitable equivalent, may be present in the composition in an amount between about 55 wt % to 99 wt %, about 60 wt % to 95 wt %, or about 65 wt % to 90 wt % by total weight of the composition. In some examples, the lactose excipient, or a suitable equivalent, may be present in an amount between 65 wt % to 70 wt %, such as about 68.5 wt % by total weight of the composition. In other examples, the lactose excipient, or a suitable equivalent, may be present in an amount between 75 and 80 wt %, such as about 76 wt % by total weight of the composition. In some cases, an amount of lactose excipient below 55 wt % (by total weight of the composition) can result in poor flowability properties.

A metal stearate excipient (such as magnesium stearate), or a suitable equivalent, may be present in the composition in an amount between about 0.1 wt % to 10 wt %, about 0.2 wt % to 5 wt %, about 0.5 wt % to 2.5 wt %, or about 0.75 wt % to 1.25 wt % by total weight of the composition. By way of further example, the composition may comprise about 1 wt % of magnesium stearate (or a suitable equivalent) by total weight of the composition.

A cellulose or cellulose derivative (such as microcrystalline cellulose), or a suitable equivalent, may be present in the composition in an amount between about 10 wt % to 30 wt %, about 15 wt % to 25 wt %, or about 18 wt % to 20 wt % by total weight of the composition. By way of further example, the composition may comprise about 19 wt % of microcrystalline cellulose (or a suitable equivalent) by total weight of the composition.

The total weight of the composition may vary and, for example, may be varied to ensure an appropriate amount and/or weight ratio of the various components is provided. For example, in some cases where a higher dosage of the active agent (e.g. ketamine hydrochloride) is desired, the total weight of the composition may be increased to ensure suitable flowability, release and/or stability properties are maintained.

The total weight of the composition may range from 25 mg to 500 mg, 50 mg to 400 mg, 75 mg to 300 mg or may be between 100 mg and 200 mg. In some instances, the total weight of the composition may be about 100 mg. In other examples, the total weight of the composition may be about 200 mg.

Accordingly, a suitable composition may comprise:
(i) ketamine hydrochloride
(ii) lactose monohydrate (e.g. spray-dried lactose monohydrate);
(iii) magnesium stearate; and
(iv) optionally microcrystalline cellulose.

Two exemplary (but not limiting) compositions are detailed in Table 1 below.

TABLE 1
Exemplary compositions comprising ketamine hydrochloride

| | Composition 1 | | Composition 2 | |
|---|---|---|---|---|
| | Weight | % by | Weight | % by |
| Material | (mg) | weight | (mg) | weight |
| Ketamine hydrochloride | 11.5 | 11.5 | 46 | 23 |
| Lactose monohydrate | 68.5 | 68.5 | 152 | 76 |
| Microcrystalline cellulose | 19 | 19 | / | |
| Magnesium stearate | 1.0 | 1.0 | 2.0 | 1.0 |
| Total | 100 | | 200 | |

Although the compositions have been shown as including ketamine hydrochloride, it will be appreciated that any form of ketamine may be used in the described compositions, including all of the ketamine types, salts, derivatives, enantiomers and/or metabolites described above.

The compositions may facilitate a manufacturing process (e.g. at scale), whilst also providing a short term (or immediate) release of ketamine and/or allowing for the stable storage of ketamine.

The compositions as described herein may be provided in an encapsulated form. For example, the disclosed composition may further comprise and/or be contained within a capsule (e.g. a hard capsule).

In some instances, the disclosed compositions may be resistant to misuse and abuse. For example, the use of a capsule (such as a hard capsule) may increase the difficulty of extracting the contents of the capsule by mechanical means (e.g. by crushing or the like).

A capsule may comprise a capsule shell. The capsule and/or capsule shell may be filled (e.g. partially or completely filled) with a composition according to this disclosure.

The capsules may be formed of any suitable material that is able to contain and/or store the composition. By way of example only, the capsules may comprise or be formed of gelatin or hydroxypropyl methylcellulose (hypromellose (HPMC)). By way of further example, the capsule may comprise or be formed of gelatin.

As stated previously, the disclosed compositions may provide for the stable storage of an active pharmaceutical ingredient for an extended period of time. In other words, active pharmaceutical agents (such as ketamine hydrochloride) which are comprised within the described compositions have been found to show good long-term storage stability. This allows the compositions to be stored under ambient conditions with minimal degradation of the active pharmaceutical agent.

As used herein, the term “good stability” may mean that an active agent comprised within the disclosed compositions is substantially stable and/or exhibits minimal or no degradation in comparison to an active agent comprised within an alternative composition. The stability of an active agent and/or its tendency to degrade is typically influenced by its chemical structure, in amongst other factors such as a surrounding environment. As such, the term “good stability” may take a different meaning when applied to different active agents.

In this instance, “good stability” may mean that an active agent comprised within the composition is stable when stored under ambient conditions (e.g. a temperature of about 25° C. and/or 60% relative humidity) for a period of at least 6 months, 12 months, 24 months or 36 months. In some cases, “good stability” may mean that an active agent comprised within the composition is stable when stored under more demanding conditions (e.g. a higher temperature/relative humidity, such as about 40° C. and/or 75% relative humidity) for a period of at least 6 months, 12 months, 24 months or 36 months.

As used herein, “stable” means that an active agent comprised within the composition may maintain at least 99%, at least 95%, at least 90%, or at least 80% of an original level of pharmaceutical activity.

Thus, the active pharmaceutical agent comprised within any one of the compositions described herein may maintain at least 99%, at least 95%, or at least 90% of an original level of pharmaceutical activity after storage for a period of at least 12, 24 or 36 months under ambient or more demanding conditions (e.g. at temperatures about or between 25° C. and 40° C. and/or relative humidities about or between 60% and 75%). This long-term stability may improve the convenience and/or accessibility of the described compositions and/or may facilitate the use of such compositions “at-home”.

The compositions described herein may be considered as rapid or immediate release formulations. In the present case, the dissolution of the various compositions was tested and the inventors determined that all compositions achieved the required level of dissolution. Suitable dissolution rates may require that a certain percentage of the active agent (in this case ketamine hydrochloride) is recovered after some predetermined time. For example, anywhere between about 1-100% of the active agent may be recovered in dissolution testing. For example, about 10%, about 20%, about 30%, about 40%, about 50%, about 60%, about 70%, about 80% about 90% or about 95% of the active agent (ketamine hydrochloride) may be recovered under dissolution testing.

The effectiveness or efficacy of any particular composition depends on the active agent dissolving in, for example, the fluids of the gastrointestinal tract. The rate of dissolution is important as this determines how much of the active agent is released and is available for use in the treatment and/or prevention of a diseases and/or disorder.

The compositions described herein may be considered as rapid or immediate release formulations. In the present case, the dissolution of the various compositions was tested and the inventors determined that all compositions achieved the required level of dissolution. Suitable dissolution rates may require that a certain percentage of the active agent (in this case ketamine hydrochloride) is recovered after some predetermined time. For example, anywhere between about 1-100% of the active agent may be recovered in dissolution testing. For example, about 10%, about 20%, about 30%, about 40%, about 50%, about 60%, about 70%, about 80% about 90% or about 95% of the active agent (ketamine hydrochloride) may be recovered under dissolution testing.

It should be noted that within the context of dissolution testing, the term “x %” means a percentage of the total amount of active agent loaded into the composition. For example any given composition may be thought of as containing (before dissolution testing) an amount equivalent to 100% of the active agent. Under dissolution testing conditions the amount of active agent released is determined and this amount is expressed as a percentage of the total amount of the active agent originally provided within the composition.

As stated, the dissolution profile of any given composition may be assessed over a predetermined period of time; for example from about 1 minute to about 60 minutes. The dissolution profile may be assessed over, for example, about 10 minutes, about 15 minutes, about 20 minutes, about 25 minutes, about 30 minutes, about 35 minutes, about 40 minutes, about 45 minutes, about 50 minutes or about 55 minutes.

In one example, an acceptable dissolution profile may be, for example, at least 75% recovery of the active agent (e.g. ketamine hydrochloride) after 30 minutes. For example, an acceptable dissolution profile may be at least 75%, at least 80%, at least 85%, at least 90% or at least 95% recovery of the active agent from the composition after 30 minutes in an aqueous solution at a temperature between 35 and 40° C. In some cases, there may be at least 75%, at least 80%, at least 85%, at least 90% or at least 95% recovery of the active agent from the composition after 30 minutes in a stirred aqueous buffer solution at pH 1.2 at a temperature of approximately 37° C.

As stated above, the compositions described herein may be considered as an immediate release formulation.

As used herein, an immediate release formulation may be a formulation that has been developed to dissolve without delaying or prolonging dissolution and/or absorption of a pharmaceutically active agent.

As used herein, the term “immediate release” may be as defined in the European Pharmacopoeia (Ph. Eur.) 9th edition. For example, an immediate release formulation as described herein may provide an in vitro dissolution of at least 75% of the active substance within 45 minutes (see, for example, the Ph. Eur. (5.17.1) recommendation for conventional release dosage forms).

As used herein, the term “immediate release” may be as defined in the United States Pharmacopoeia (USP) or as defined by the US Food and Drug Administration (FDA) (see, for example, “Dissolution Testing and Acceptance Criteria for Immediate-Release Solid Oral Dosage Form Drug Products Containing High Solubility Drug Substances, Guidance for Industry, Biopharmaceutics, August 2018 and also US Food and Drug Administration Centre for Drug Evaluation and Research Guidance for Industry: Dissolution Testing of Immediate Release Solid Oral Dosage Forms. Silver Spring; MD: 1997). For example, an immediate release formulation as described herein may provide for the release of more than 80% of the drug substance within 30 minutes.

The compositions described herein may provide rapid or very rapid release formulations.

In the framework of BCS (Biopharmaceutics Classification System)-based biowaiver (a surrogate for in vivo bioequivalence), a very rapid release may be defined as when 85% of the labelled content is dissolved within 15 minute. Rapid release may be when 85% of the labelled content is dissolved within 30 minutes.

The compositions may be intended for oral administration. As such, the compositions may be suitable and/or be formulated for oral administration. In particular, the compositions may be administered orally and may facilitate an immediate or short-term release of ketamine (as described above).

The compositions described herein have a pharmacokinetic profile which differs markedly from many other (prior art) ketamine formulations. Ketamine that is formulated for nasal, IV or IM administration, tends to be absorbed into the bloodstream and so avoid early first pass metabolism. In contrast, when administered orally, the bioavailability of ketamine is much lower and is subject to first pass metabolism into a number of different metabolites. Further, the composition avoids the metallic taste which is characteristic of some formulations. In addition, ketamine metabolites are postulated to be important in eliciting ketamine's anti-depressant, anti-suicidal effects and pain relieving effects.

The compositions provided by this disclosure may have a variety of clinical applications.

As such, a further aspect of this disclosure provides one or more of the compositions described herein, for use in medicine or for use as a medicament. For example, the various compositions may be applied (i.e. may be for use in) the treatment or prevention of one or more of the following indications.

One or more of the compositions described herein may find use in the treatment, prevention, control and/or management of pain.

It should be noted that the term “pain” embraces any pain associated with mental health, neurological, dermatological disorders, psychiatric disorders, wounds, minor surgical procedures, peri-operative pain; inflammatory disorders, cancer, asthma, chronic obstructive airway disease, painful procedures (for example in emergency departments) and emergency surgery in field conditions in trauma situations or war zones. The compositions described herein may also be for use in the management, treatment or prevention of post-surgery pain; opioid use; hyperalgesia; neuropathic pain; ischemic limb pain; peripheral neuropathies; complex pain; complex regional pain syndrome type I and type II; phantom limb pain; nerve damage pain; post-herpetic neuralgia; Chronic ischemic monomelic neuropathy from critical limb ischemia; peripheral ischemic neuropathies; ischemic neuropathies associated with diabetes; complex pain across indications that are both inflammatory and neuropathic; clinical features across indications that include hyperalgesia, allodynia, central sensitisation, wind-up pain, dysaesthesias, paraesthesia, paroxysmal pain, and pain inadequately controlled by existing drug treatments. The compositions of this disclosure may also be for use as pain relief in patients who require elevated blood pressure, maintenance of airway reflexes/skeletal muscle tone, or who may benefit from Ketamine's bronchodilatory properties.

One or more of the compositions described herein may also be used for the treatment, prevention, control and/or management of certain neurological or psychlogical disorders. Neurological disorders that may be treated by any of the compositions described herein may include, for example, various forms of palsy, palsy/writing and inflammation associated with Parkinson's disease, the side-effects of Parkinson's disease drugs, multiple sclerosis, Parkinson's plus diseases, migraine, Alzheimer's disease, epilepsy, dyskinesia e.g. L-dopa-Induced Dyskinesias and movement disorders.

One or more of the compositions described herein may find use in the treatment, prevention, control and/or management of a mental health disorder, mood disorder or a psychiatric disorder. Mental health, mood disorder or psychiatric disorders that may be treated by any of the compositions described herein may include, for example, a depressive disorder.

A further aspect provides a method of treating a mental health, mood or psychiatric disorder, said method comprising administering a composition as described herein to a subject in need thereof (for example a subject suffering from or predisposed to, any of the mental health disorders, mood disorder or psychiatric disorders described herein).

Yet a further aspect provides a use of a composition as described herein in the manufacture of a medicament for treating, preventing, controlling and/or managing a mental health disorder, mood disorder or psychiatric disorder (e.g. a depressive disorder).

When delivered intravenously, ketamine is known to elicit a rapid antidepressant response. However, an injectable form of an active agent often requires costly hospital stays, or appointments, and so is not always immediately accessible to a subject in need thereof. In contrast, and without being bound by theory, ketamine comprised within the various compositions described herein may provide a convenient and/or accessible treatment option for a subject in need thereof. In particular, as stated previously, the compositions may be suitable for oral administration. Thus, the described compositions may be suitable for use “at home” and/or without the need for hospital stays or interventions from a medical practitioner. In particular, the described compositions may find particular application in complementing an existing course of treatment and/or in the treatment and/or control of acute episodes of depression and/or suicidal ideation associated with such.

When administered orally, ketamine is subject to first pass metabolism into a number of different metabolites (e.g. ketamine is metabolized in the liver via N-demethylation and ring hydroxylation pathways and the main metabolites are norketamine and its hydroxylated derivatives (hydroxynorketamine). Ketamine metabolites are postulated to be important in eliciting ketamine's anti-depressant and anti-suicidal effects and, for example, metabolites such as 2S,6S-Hydroxynorketamine hydrochloride and cis-6-Hydroxynorketamine hydrochloride are known antidepressants. Thus, without being bound by theory, any ketamine comprised within the compositions described herein may elicit an antidepressant effect directly in itself or by way of its metabolites following an oral administration.

A depressive disorder that may be treated, prevented, controlled and/or managed by any one of the described compositions may include, but is not limited to, suicidal ideation, suicidality, major depressive disorder, treatment resistant depression, dysthymia, post-partum depression, atypical depression (AD), melancholic depression, psychotic major depression, catatonic depression, double depression, depressive personality disorder, depressive disorder not otherwise specified, premenstrual dysphoric disorder, seasonal affective disorder (SAD), recurrent brief depression and minor depressive disorder.

In particular, one or more of the compositions described herein may also be useful in the treatment, prevention, control and/or management of “treatment-resistant depression”.

Treatment-resistant depression (TRD) may be defined as an episode of depression (or a condition or disease associated therewith) for which one or more treatment interventions has failed-particularly where the treatment intervention(s) have (or has) failed despite where the intervention is used for a sufficient duration of time and at adequate dose. By way of example, TRD may be defined as (i) failure to respond to one or more (drug or non-drug) anti-depressant therapy; or (ii) failure to respond to two or more currently available antidepressants of adequate dose and duration in the current episode of depression. Thus, in such cases, a composition of the disclosure may be administered at a suitable dosage and for a suitable time period to a subject suffering from an episode of depression who has failed to respond to one or two or more other anti-depressant treatments.

Additionally or alternatively, the various compositions described herein may each be used in the treatment, prevention and/or control of suicidal ideation and behaviour associated with a mental health disorder or a psychiatric disorder. In particular, the various compositions described herein may find use in the treatment, prevention and/or control of suicidal ideation or behaviour associated with a depressive disorder (e.g. treatment resistant depression).

In particular, the compositions may find application in the treatment, prevention and/or control of acute episodes of suicidal ideation in a subject with an underlying mental health disorder or psychiatric disorder. As used herein, an acute episode of suicidal ideation or behaviour means a sudden and/or severe onset of a suicidal episode.

Without being bound by theory, the compositions described herein may allow the rapid release of the active agent in vivo, e.g. after oral administration. Consequently, this may be useful in providing a fast-acting treatment and/or readily available treatment option for a subject suffering from a suicidal episode and/or an acute episode of depression. In particular, the described compositions can provide the stable storage of the active agent for extended periods of time and/or can be administered without the need for intervention from a medical practitioner (facilitating “at-home” use and/or avoiding the costs of a hospital stay).

In other words, a subject may be administered ketamine orally (e.g. by way of a composition as described herein) as a primary treatment option. Such primary treatment options include, but are not limited to, the treatment, prevention, control and/or management of a mental health disorder, mood disorder or a psychiatric disorder, in particular the treatment and/or control of acute episodes of depression and/or suicidal ideation associated with such. In other examples, a subject with suspected depression may be administered ketamine orally while waiting to be tested or assessed.

The compositions provided by this disclosure may be used alone or in combination other medications and/or treatments.

As used herein, “in combination with” may indicate that the delivery of two or more therapeutic agents to a subject takes place either concurrently or at different times. For example, one therapeutic agent may be administered to a subject in combination with another by administering both agents to the subject concurrently, such as in a single composition or in separate compositions that are administered to the subject simultaneously (e.g. by different routes of administration). In another example, one therapeutic agent may be administered to a subject in combination with another by first administering to the subject one therapeutic agent and subsequently administering the other therapeutic agent, either by the same or different route of administration. In other words, the use of the compositions “in combination” with other medications and/or treatments may mean that the compositions are administered concurrently with, alongside, before or after any other medications and/or treatments.

In particular, any of the compositions described herein may be used in combination with any other anti-depressant medications and/or psychotherapy. Other anti-depressants that may be used in combination with the described compositions may include, but are not limited to, selective serotonin reuptake inhibitors (SSRIs), serotonin-noradrenaline reuptake inhibitors (SNRIs), noradrenaline and specific serotonergic antidepressants (NASSAs), tricyclic antidepressants (TCAs), monoamine oxidase inhibitors (MAOIs), and other ketamine formulations (e.g. injectable ketamine).

In a yet further aspect of the disclosure, there is provided ketamine for use in treating a depressive disorder in a subject in need thereof, wherein the ketamine is administered orally and wherein the ketamine is administered in combination with a second anti-depressant agent.

There is also provided a method for treating a depressive disorder in a subject in need thereof, by orally administering a therapeutically effective amount of ketamine, wherein the ketamine is administered in combination with a second anti-depressant agent.

There is further provided a use of ketamine in the manufacture of a medicament for use in treating a depressive disorder in a subject in need thereof, wherein the ketamine is administered orally and wherein the ketamine is administered in combination with a second anti-depressant agent.

In other words, the ketamine (which may optionally be comprised within one of the compositions described herein) may be administered to complement and/or supplement a new or existing course of anti-depressant treatment based or involving the use of a second anti-depressant agent (e.g. as an “add-on” or “top-up” treatment).

In such cases, the ketamine may be considered as a secondary treatment that may be prescribed for the purposes of early augmentation and support or intervention, e.g. in those cases where the primary treatment (which is based on the second anti-depressant agent) has a slower onset in terms of the desired therapeutic effect.

As stated previously, the term “ketamine” in this aspect (and indeed all of the aspects described herein) may embrace any form of ketamine, including all of the ketamine types, salts, derivatives, enantiomers and/or metabolites described above.

The second anti-depressant agent may be any of those as defined herein. For example, the ketamine may be administered in combination with a second anti-depressant agent selected from the group consisting of selective serotonin reuptake inhibitors (SSRIs), serotonin-noradrenaline reuptake inhibitors (SNRIs), noradrenaline and specific serotonergic antidepressants (NASSAs), tricyclic antidepressants (TCAs), monoamine oxidase inhibitors (MAOIs), and other ketamine formulations (e.g. injectable ketamine). In some cases, the ketamine may be administered in combination with a selective serotonin reuptake inhibitor.

A subject suffering from a depressive disorder and who is beginning or undergoing treatment with a second anti-depressant agent may still experience instability in their levels of depression, relapses in depression and/or acute episodes of depression and/or suicidal ideation and/or behaviour. The oral administration of ketamine (e.g. in one of the compositions disclosed herein) may be used to treat, control and/or prevent such instability, relapses and/or acute episodes of depression and/or suicidal ideation and/or behaviour.

For example, a subject already being treated with one or more of the second anti-depressant agents (as defined above) may go through periods of instability and/or suffer an acute depressive episode and/or an acute episode of suicidal ideation and/or behaviour. The oral administration of ketamine (e.g. in one of the compositions described herein) may be useful in assisting in the control, treatment, prevention and/or management of such periods and acute episodes.

Without being bound by theory, the oral administration of ketamine can elicit an anti-depressive effect more rapidly than many other types of anti-depressant agent. For example, at some dosages, the oral administration of ketamine can provide an anti-depressive effect within one week (approximately 168 hours). In contrast, many other types of anti-depressants can take many weeks to have the desired effect.

By way of further example, where a subject in need thereof begins a course of treatment based on the second anti-depressant agent, there may be a period of time before the treatment has the intended therapeutic effect and/or is considered established. In other words, there may be a time lag between initiation of a treatment with the second anti-depressant agent and the subject showing an improvement in their condition. In such cases, subjects suffering from a depressive disorder may experience instability, relapses and/or acute episodes of depression and/or suicidal ideation and/or behaviour in the period following initiation of the treatment with the second anti-depressant agent. Accordingly, the oral administration of ketamine may be particularly useful during an initial period of treatment with the second anti-depressant agent (e.g. until such time as the second anti-depressant agent has elicited the desired therapeutic effect).

The ketamine (which may be comprised within a composition as described herein) may be administered once (e.g. as a one-off treatment) or may be administered multiple times over a period of time (e.g. as part of a treatment regimen). A suitable treatment regimen may vary and/or be determined by the desired therapeutic effect (e.g. the indication being treated).

For example, the ketamine may be administered during a period of instability, relapse, and/or acute depressive episode and/or a period where the subject is suffering from suicidal ideation and/or behaviour associated with a depressive disorder. The ketamine may be administered one, two, three, four or more times during this period and/or may be administered until the desired therapeutic effect has been elicited (e.g. the subject reports an improvement in their level of depression and/or their level of suicidal ideation and/or behaviour).

The ketamine (which may optionally be comprised in one of the compositions described herein) may be administered to the subject for a period of at least 1, 2, 3, 4, 5 or 6 weeks or up to 10, 11, or 12 weeks.

This period may be any period of time following administration of the second anti-depressant agent and/or initiation of a treatment regimen comprising multiple administrations of the second anti-depressant agent. In some instances, this period may start upon or after the initiation of a treatment regimen involving and/or based on the administration of the second anti-depressant agent. By way of further example, this period may run concurrently with the initial period of a treatment regimen comprising multiple administrations of the second anti-depressant agent.

The second anti-depressant agent may be an injectable ketamine formulation. Injectable ketamine formulations may be administered to a subject intravenously, intramuscularly, subcutaneously, sublingually or nasally. Consequently, the oral administration of ketamine (e.g. comprised within a composition as disclosed herein) may be used in combination with a primary treatment in which ketamine is administered to a subject in need thereof by another route (e.g. intravenously or intramuscularly or subcutaneously or sublingually or nasally).

In particular, in such cases, a primary course of treatment may involve a subject being administered ketamine (e.g. injectable ketamine) intravenously, intramuscularly, subcutaneously, sublingually or nasally in a clinic or hospital setting. The primary course of treatment may comprise one or multiple administrations of ketamine (e.g. injectable ketamine) via an intravenous or intramuscular or subcutaneous or sublingual or nasal route. The oral administration of ketamine (e.g. in one of the compositions disclosed herein) may be used to complement and/or supplement this primary course of treatment and/or may provide an accessible and/or convenient treatment option (e.g. that may be suitable for use “at home” by way of a “maintenance therapy” or “top-up” therapy) without requiring to re-attend the clinic.

In each of the various aspects described herein, the compositions may comprise a therapeutically effective amount and/or a unit dosage amount of the active agent (e.g. ketamine). In other words, each composition may be designed to provide a unit dosage amount to a subject in need thereof.

The suitable amount and/or unit dosage may be determined in accordance with a desired therapeutic effect. For instance, the suitable amount and/or unit dosage may vary dependent upon the desired therapeutic effect of the composition. By way of example, a suitable amount or unit dosage may be between 40 mg and 400 mg.

The disclosure also provides a treatment regimen by which ketamine (e.g. comprised within a composition of this disclosure) is administered to a subject so as to treat, prevent, control and/or manage a mental health disorder, mood disorder or a psychiatric disorder as described herein.

The treatment regimen may vary dependent upon the desired therapeutic effect. In some cases, the treatment regimen may involve the administration of a unit dosage amount of ketamine once a day. In other cases, the treatment regimen may comprise the administration of a unit dosage amount of ketamine once a week or multiple times per week (e.g. 2, 3, 4 or 5 times per week, such as two or three times per week). The duration of the treatment regimen may range from 1 week to 12 weeks, or from 1 week up to 3 months.

Successful treatment of any of the above conditions (that is the successful treatment, prevention, control or management of any one of the conditions or disorders stated above) may be gauged by changes in the level of depression and/or changes in an assessment of suicidal ideation and behaviour.

A level of depression may be assessed using a depression rating scale such as the Hamilton Depression Rating Scale, Montgomery-Åsberg Depression Rating Scale (MADRS), Raskin Depression Rating Scale, Beck Depression Inventory, self-report inventory Geriatric Depression Scale (GDS)e, Zung Self-Rating Depression Scale Patient Health Questionnaire (PHQ) Patient Health Questionnaire-9 (PHQ-9) Primary Care Evaluation of Mental Disorders (PRIME-MD) Patient Health Questionnaire. The Beck Depression Inventory; The Clinically Useful Depression Outcome Scale, The Inventory of Depressive Symptomatology, The Mood and Feelings Questionnaire, The Quick Inventory of Depressive Symptoms.

As used herein, an instability in levels of depression, a relapse in depression and/or an acute depressive episode may refer to (or embrace) a clinical situation wherein a subject experiences a change (e.g. an increase) in their level of depression as assessed by any of the rating scales noted above.

In terms of suicide, suicidal ideation and behaviour may be assessed using the suicidal ideation and behaviour assessment tool (SIBAT) or the suicidality elements of Montgomery-Åsberg Depression Rating Scale (MADRS) or additional scales that contain specific suicidality assessments.

In a further aspect, the present disclosure also provides a method of making any one of the disclosed compositions.

A composition prepared according to a method described herein may use or exploit an amount of one or more of the active agents and excipients as described above in the first aspect. In terms of amounts, the method of manufacture may utilise any of the amounts or quantities defined as for the first aspect.

The method may comprise contacting, combining, mixing and/or blending one or more excipients together with the active agent (e.g. ketamine hydrochloride) to provide the composition.

The step of combining, contacting, mixing and/or blending the excipients together with the active agent may comprise combining, contacting, mixing and/or blending one or more of the excipients with the active agent to provide a pre-mixed or pre-blended composition. One or more further excipients may then be added to the pre-mixed or pre-blended composition and further mixing and/or blending is carried out in order to provide the final composition. In some instances, the pre-mixed or pre-blended composition may comprise the active agent, together with one or more excipients selected from the group consisting of fillers, diluents and flow aids. The one or more excipients that may be added to the pre-mixed or pre-blended composition may comprise a lubricant.

By way of example only, a method of providing the disclosed compositions may comprise the following steps:
(a) mixing and/or blending one or more excipients selected from the group consisting of fillers, diluents and compression aids with the active agent (such as ketamine hydrochloride) to provide a pre-mixed and/or pre-blended composition; and
(b) mixing and/or blending a lubricant with the pre-mixed and/or pre-blended composition to provide the composition.

In particular, the method may comprise:
(a) mixing and/or blending lactose (such as lactose monohydrate) and optionally a cellulose derivative (such as microcrystalline cellulose) with the active agent (such as ketamine hydrochloride) to provide a pre-mixed and/or pre-blended composition; and
(b) mixing and/or blending a metal stearate (such as magnesium stearate) with the pre-mixed and/or pre-blended composition to provide the composition.

One or more, or all of the components of the composition (e.g. the active agent and excipients) may be sieved prior to a mixing and/or blending step.

The composition may be used to fill (e.g. partially or completely fill) a capsule to provide an encapsulated formulation. Thus, the method may further comprise a step of filling a capsule with the composition.

The step of filling a capsule may be automated. For example, the capsule may be filled with a composition on an automated capsule filling machine.

As described previously, there are challenges associated with filling capsules with a pharmaceutical composition. In particular, it is important that the filled capsules are produced with good weight uniformity. In addition to all of this, any composition must show good blending properties (to provide a homogenous mixture), whilst also being compatible with the active pharmaceutical ingredient in question to the extent that the active pharmaceutical ingredient shows good stability upon storage and/or maintains the desired release properties.

The present inventors have identified that the compositions disclosed herein show good flowability properties and so may be particularly suitable for use in a capsule filling method (e.g. an automated capsule filling method). In particular, it is believed that the flowability properties not only offer an efficient filling of capsules but provide capsules with a good weight uniformity. Without being bound by theory, it is hypothesised that the flow properties of a composition (e.g. a powder composition) are closely correlated with weight variability in a capsule filling process. Thus, the compositions with good flowability have been found to provide filled capsules with good weight uniformity and/or minimum weight variability.

In a further aspect, there is provided a use of one or more of the compositions described herein to fill a capsule. For example, the compositions may be used to fill one or more capsules in an automated capsule filling process.

In one teaching, the present disclosure provides ketamine (as described herein) and/or any of the compositions described herein (also comprising ‘ketamine’ as described herein) for use in treating, preventing or controlling any one or more of the disclosed depressive disorders-including, for example, the treatment, control or prevention of treatment resistant depression (TRD).

The disclosure also provides methods of treating, controlling or preventing the disclosed depressive disorders-including, for example, treating, controlling or preventing TRD, said method comprising administering a subject in need thereof ketamine (as described herein) or a composition comprising the same.

In one teaching, a (medical) use or a method of treatment provided by this disclosure, may comprise the administration of ketamine via two or more different routes. For example, ketamine may first be used or administered intravenously, subcutaneously, sublingually or intra-nasally. Ketamine may then be used or administered orally. The different routes may be administered at the same time, concurrently or separately and at different times.

Accordingly, the disclosure provides ketamine for use in treating, controlling or preventing any one or more of the disclosed depressive disorders-including, for example, the treatment, control or prevention of TRD, wherein the ketamine first is administered intravenously, subcutaneously, sublingually or intra-nasally and then orally. Moreover, a method of treating, controlling or preventing any of the disclosed depressive disorders (including, for example, treating, controlling or preventing TRD), may comprise administering a subject in need thereof a first dose of ketamine administered intravenously, subcutaneously, sublingually or intra-nasally and a second dose of ketamine administered orally.

When ketamine (for example s-ketamine) is to be administered or used subcutaneously, the ketamine may be used at a dose of 0.1 mg/kg to 10 mg/kg (for example at a dose of 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg or 9 mg/kg). For example, subcutaneous ketamine may be administered at a dose of between 0.5 mg/kg and 1.0 mg/kg.

When ketamine (for example s-ketamine) is to be administered or used subcutaneously, the ketamine may be administered once a day, once a week, twice a week, three times a week, four times a week, five times a week, six times a week or seven times a week.

For example, subcutaneous ketamine may be administered once a week at a dose of between 0.5 mg/kg and 1.0 mg/kg.

Subcutaneous ketamine may be administered at the required dose and frequency for 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks or 40 weeks.

For example, subcutaneous ketamine may be administered once a week for 3 to 4 weeks, wherein each administered dose comprises between 0.5 mg/kg and 1.0 mg/kg ketamine.

When ketamine is to be administered or used orally, the ketamine may be used at a dose of 10 mg/day to 500 mg/day. The required dose may be made up of a single amount of multiple smaller amounts amounting to the required daily dose. For example oral ketamine may be administered at a dose of 10 mg, 20 mg, 30 mg, 40 mg, 50 mg, 100 mg, 120 mg, 150 mg, 160 mg, 170 mg, 180 mg, 200 mg, 220 mg, 240 mg, 300 mg, 350 mg, 400 mg or 450 mg.

Oral ketamine may be administered together with any subcutaneous/IV ketamine dose and/or separately and at a different time.

The oral dose may be administered at the required dose and frequency for 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks or 40 weeks.

In one teaching, the frequency of any oral dose may be increased. For example, an initial oral dose may be administered once a week whereas a second or subsequent dose may be administered more frequently, for example twice a week, three times a week or more.

In any medical use or method of treatment described herein, ketamine may first be administered subcutaneously at any of the stated doses/frequencies/duration and then orally at any of the stated doses/frequencies/duration(s).

In any medical use or method of treatment described herein, ketamine may first be administered subcutaneously at a dose of 0.5 mg to 1.0 mg/kg for the required duration and frequency and then orally at a dose of 120 mg-240 mg/day for the required duration. The ketamine may be administered as multiple smaller doses to the maximum stated daily dose. For example, a daily dose of 120-240 mg ketamine may be administered as 3-6 doses of 40 mg ketamine per day.

For example in any medical use or method of treatment described herein, ketamine may first be administered subcutaneously once a week for three-four weeks wherein each administered dose comprises 0.5 mg to 1.0 mg/kg ketamine and then once a week orally at a dose of 120 mg-240 mg/day. The oral dose may be administered once a week for 1-7 weeks and then increased to, for example twice a week at weeks 7-8.

Any first (subcutaneous/intravenous) ketamine dose may be administered concurrently with any oral dose. Alternatively any first ketamine dose (administered subcutaneously or intravenously) may be separated from a second (oral) ketamine dose by a period of 1 day-14 days. For example the first and second ketamine doses may be separated by a period of, 2 days, 3 days, 4 days, 5 days, 6 days, 7 days, 8 days, 9 days, 10 days, 11 days, 12 days or 13 days. By way of example, in a medical use or method of treatment described herein, ketamine may first be administered subcutaneously at a dose of 5 mg to 1.0 mg/kg (applying any of the frequencies/durations described herein) and then 4 days later, orally at a dose of 120 mg-240 mg/day (again applying any of the frequencies/durations described herein).

In any medical use or method of treatment described herein, ketamine may first be administered intravenously at any of the stated doses/frequency and then orally at any of the stated doses.

Without wishing to be bound by theory, it is submitted that the combined use of two or more different routes of administration, results in a ketamine-based treatment for the disclosed depressive disorders, including, for example, conditions such as treatment resistant depression with a low rate of adverse events that are were moderate and self-limiting. Moreover, any treatment for severe and chronic TRD (characterised by high levels of treatment refractoriness, including for ECT) represents a major clinical gain. Oral ketamine used alone or together with (e.g. as an adjunct to) a subcutaneous/intravenous dose, is a safe, well-tolerated, and potentially effective treatment for patients with chronic and severe TRD combined with ketamine subcutaneous and/or IV, even after unsuccessful ECT.

It should be noted that a change in depressive symptom severity can be expressed as a change in total MADRS score (for example between pre-treatment and the following treatment). The Visual Analogue Scale (VAS) is a scale to measure mood symptoms. In this regard, a decrease in MADRS and VAS appears to be related to the combined use of IV and/or subcutaneous doses of Ketamine with oral ketamine (as described herein).

The current data also shows that patients who stop an initial IV or subcutaneous-based ketamine treatment respond very well to oral ketamine, especially oral ketamine administered twice a week at a dose of between 160-240 mg.

Disclosed herein is ketamine for use in maintaining remission from a depressive disorder.

A depressive disorder may be brought into remission by a combined ketamine treatment comprising the use of a subcutaneous/IV ketamine dose and an oral ketamine dose. The dose, frequency and duration of any subcutaneous, intravenous and/or oral ketamine dose is in accordance with the disclosure set out above.

It has been shown that a combined treatment comprising the use of a subcutaneous/IV ketamine dose and an oral ketamine dose (administered as per the disclosure above) can bring a depressive disorder into remission and that the period of remission can be maintained and/or extended solely with the use of an oral dose of ketamine (again administered as per any of the doses set out herein).

A treatment effective dose of ketamine may be any dose (at any amount/frequency and for any duration) of subcutaneous/intravenous or oral administered ketamine as described herein. Likewise, a remission maintaining or remission maintaining dose of subcutaneous/intravenous or oral administered ketamine may be any dose (at any amount/frequency and for any duration) of ketamine as described herein.

A subject to be administered any of the subcutaneous/intravenous or oral administered doses of ketamine described herein (including any of the combined treatments described herein) may be any subject:
(i) with a depressive disorder (including TRD); or
(ii) susceptible or predisposed to a depressive disorder (including TRD); or
(iii) suspected of suffering from a depressive disorder (including TRD); or
(iv) being treated for a depressive disorder (including TRD).

It should be understood that, where appropriate, the term “comprising” may encompass the terms “consisting essentially of” and “consisting of”. As used herein, the term “subject” may refer to a mammalian subject (e.g. a human subject). Additionally, each of the definitions and embodiments provided in this specification applies to each aspect of this disclosure.

### DETAILED DESCRIPTION

The present disclosure will now be described in more detail with the following non-limiting examples.

Investigations were carried out in order to identify ketamine-based compositions that would be suitable to be formulated into capsules. In particular, the inventors were looking to identify compositions that would be compatible with an automated capsule filling process, whilst also maintaining good stability and desirable release profiles.

The results of these investigations are summarised below.

### Initial Blend Experiments

A number of formulations (10 g batch size) were prepared by blending materials together in a plastic container. Bulk and tapped density and compressibility index were calculated for each blend. Details are given in Table 2 below.

TABLE 2
Initial Blending Experiments

| Formulation | 1 | 2 | 3 | 4 | 5 | 6 |
|---|---|---|---|---|---|---|
| Strength (mg) | 10 | 10 | 40 | 40 | 40 | 40 |
| API quantity | 1.15 | 1.15 | 4.60 | 4.60 | 2.30 | 1.53 |
| (g) | | | | | | |
| Lactose | 6.85 | 8.85 | 5.40 | 3.40 | 7.70 | 8.47 |
| Monohydrate | | | | | | |
| quantity (g) | | | | | | |
| Avicel PH101 | 1.90 | — | — | 1.90 | — | — |
| quantity (g) | | | | | | |
| Magnesium | 0.10 | — | — | 0.10 | — | — |
| stearate | | | | | | |
| quantity (g) | | | | | | |
| Fill weight | 100.0 | 100.0 | 100.0 | 100.0 | 200.0 | 300.0 |
| (mg) | | | | | | |
| Poured Density | 0.53 | 0.61 | 0.53 | 0.43 | 0.56 | 0.59 |
| (g/ml) | | | | | | |
| Tapped | 0.69 | 0.74 | 0.77 | 0.67 | 0.74 | 0.74 |
| Density (g/ml) | | | | | | |
| Compressibility | 23.7 | 18.2 | 31.6 | 34.8 | 25.0 | 20.6 |
| Index | | | | | | |
| Prediction of | Pass- | Fair | Poor | Very | Pass- | Pass- |
| Flow properties | able | | | Poor | able | able |

The Compressibility Index results for formulations 1 and 2 indicated that the flow properties of these compositions were likely to be suitable for an automated capsule filling process. However, the relatively higher Compressibility Index results for the 40 mg strength (formulations 3 and 4) indicated that flow properties were unlikely to be suitable for filing on an automated capsule machine.

In order to improve the flow properties, blends containing a greater proportion of lactose monohydrate were prepared in 200 mg and 300 mg fill weights (formulations 5 and 6). (These formulations contained the appropriate amount of ketamine hydrochloride to provide a dosage amount of 40 mg ketamine in each capsule). The improved Compressibility Index values for these formulations indicated that suitable flow properties could be achieved for compositions comprising a 40 mg dosage amount of ketamine by increasing the fill weight of the capsule to between 200 and 300 mg and the proportion of lactose monohydrate present in the composition. It was observed that compositions comprising over 60 wt % of lactose monohydrate showed suitable flowability properties.

### Filling of Capsules

In order to establish possible capsule sizes, Formulations 1, 4, 5 and 6 were hand filled into capsules. The results are shown in Table 3 below.

TABLE 3
Capsule Filling

| Formulation | 1 | 4 | 5 | 6 | 6 |
|---|---|---|---|---|---|
| Strength (mg) | 10 | 40 | 40 | 40 | 40 |
| Capsule Size | 2 | 2 | 2 | 2 | 1 |
| Nominal Fill | 100.0 | 100.0 | 200.0 | 300.0 | 300.0 |
| Weight (mg) | | | | | |
| Weight range | 97.6- | 98.1- | 195.7- | Max 230 mg | 294.4- |
| obtained (mg) | 103.1 | 102.8 | 203.6 | obtained | 308.4 |
| Suitable size | Yes | Yes | Yes | No | Yes |

Size 2 capsules were found to be suitable for fill weights of up to 200 mg.

The smaller capsule size was deemed preferable (e.g. in terms of patient compliance) and so this capsule size was selected for further investigations for both the 10 mg and 40 mg dosage compositions.

### Further Formulation Investigations

The proposed capsule formulations are set out in table 4 below. Size 2 gelatin capsules (with a nominal weight of approximately 61±4 mg) were selected for both the 10 mg and 40 mg dosage capsule formulations. (A 10 mg dosage formulation comprising 10 mg of ketamine and a 40 mg dosage formulation comprising 40 mg ketamine respectively).

TABLE 4
Proposed capsule formulations

| | Quantity per | Quantity per |
|---|---|---|
| | 10 mg Capsule | 40 mg Capsule |
| Material | (mg) | (mg) |
| Ketamine Hydrochloride | 11.50 | 46.00 |
| Lactose Monohydrate Spray | 68.50 | 152.00 |
| Dried (Supertab 11SD) | | |
| Microcrystalline cellulose | 19.00 | — |
| Magnesium stearate | 1.00 | 2.00 |
| Capsule size 2 White/White | 1 | — |
| Capsule size 2 Blue/White | — | 1 |

Blends containing the equivalent of approximately 440 capsules of each strength were prepared by blending the API (ketamine hydrochloride) and excipients (except magnesium stearate) for 10 minutes in a drum blender. Magnesium stearate was sieved onto the blends, which were then blended for a further 2 minutes.

The final blends were filled into capsules using a Bonapace IN-CAP automatic capsule filling machine.

### 10 mg Capsules

The prepared blend flowed satisfactorily on the machine and produced capsules with a good weight uniformity. Weights obtained are given in Table 5 below.

TABLE 5
Weights of 10 mg Capsules (28 individual capsules)

| Target (mg) | Range (mg) | Mean (md) | % of nominal |
|---|---|---|---|
| 162.6 | 157.7-161.4 | 160.1 | 98.7 |
| | (96.9-99.3%) | | |

### 40 mg Capsules

The prepared blend flowed well on the machine and produced capsules with a good weight uniformity. Weights obtained are given in Table 6 below.

TABLE 6
Weights of 40 mg Capsules (20 individual capsules)

| Target (mg) | Range (mg) | Mean (mg) | % of nominal |
|---|---|---|---|
| 282.6 | 256.3-262.5 | 259.2 | 98.9 |
| | (97.6-100.0%) | | |

Both 10 mg and 40 mg dosage formulations showed satisfactory flow and filling properties and were therefore deemed suitable for manufacture using an automatic capsule filling machine.

### Scale-Up Manufacturing Trials

### General Method A

Ketamine Hydrochloride, Supertab 11SD and optionally Avicel PH1010 were sieved through an 850μ screen into a blending drum.
Materials were blended for 10 minutes.
Magnesium stearate was sieved through a 500μ screen on to the above blend and all materials were blended for a further 3 minutes.
The resulting blends appeared uniform with no obvious lumps present.
The blend was filled into size 2 capsules using the Bonapace IN-CAP capsule filling machine.

### 10 mg Stability Batch

A 300 g blend (equivalent to 3000 capsules) was prepared in accordance with the procedure outlined in General Method A.

The blends flowed well into the dosing drum and good plugs were formed. The final yield was approximately 1900 capsules. Capsule weights that were obtained during set up and encapsulation are given in Table 7 below.

TABLE 7
Mean weights of 10 capsules during set up
and encapsulation (limits 156.1-166.1 mg)

| Elapsed Time (min) | Mean Capsule weight (10 capsules) (mg) |
|---|---|
| Set Up 1 | 157.3 |
| Set Up 2 | 157.9 |
| Set Up 3 | 158.2 |
| 0 | 159.6 |
| 15 | 161.9 |
| 33 | 162.3 |
| 47 | 164.7 |
| 67 | 164.8 |
| 84 | 164.7 |
| 99 | 162.8 |
| End of Run | 160.5 |

The weight uniformity of the resulting capsules was good and all weight measurements taken during the encapsulation run complied with the specification.

Finished capsules were packed into 100 mg HDPE bottles with tamper evident caps (100 capsules per bottle) and placed on stability test.

### 40 mg Stability Batch

A 600 g blend (equivalent to 3000 capsules) was prepared in accordance with the procedure outlined in General Method A.

The blends flowed well into the dosing drum. The final yield was approximately 2100 capsules. Capsule weights that were obtained during set up and encapsulation are given in Table 8 below.

TABLE 8
Mean weights of 10 capsules during set up
and encapsulation (limits 251.1-271.2 mg)

| Elapsed Time (min) | Mean Capsule (10 capsules) (mg) |
|---|---|
| Set Up 1 | 259.7 |
| Set Up 2 | 261.5 |
| Set Up 3 | 262.1 |
| 0 | 262.1 |
| 16 | 264.9 |
| 32 | 268.0 (Machine adjustment made) |
| 33 | 262.5 |
| 50 | 262.0 |
| 66 | 262.9 |
| 83 | 261.5 |
| End of run | 260.4 |

The weight uniformity of the resulting capsules was good and all weight measurements taken during the encapsulation run complied with the specification.

Finished capsules were packed into 100 mg HDPE bottles with tamper evident caps (100 capsules per bottle).

### Stability Studies

The stability of 10 mg and 40 mg dosage capsules was investigated. The results are shown in tables 9 to 12 below.

TABLE 9
stability data of 10 mg dosage formulations stored at
25° C./60% RH (relative humidity) (% LC—% of
label content; NMT—not more than; LOQ—level of quantification)

| Duration | Ketamine Content (% LC) | |
|---|---|---|
| Stability | 90.0-110.0% of nominal | Total degradation products |
| Specification | content | NMT 0.5% |
| Initial | 101.8 | <LOQ |
| 4 weeks | 95.6 | <LOQ |
| 8 weeks | 103.9 | <LOQ |
| 13 weeks | 102.2 | <LOQ |
| 26 weeks | 102.2 | <LOQ |
| 52 weeks | 99.7 | <LOQ |
| 108 weeks | 102.2 | <LOQ |

TABLE 10
stability data of 10 mg dosage formulations stored at
40° C./75% RH (relative humidity) (% LC—% of
label content; NMT—not more than; LOQ—level of quantification)

| Duration | Ketamine Content (% LC) | |
|---|---|---|
| Stability | 90.0-110.0% of nominal | Total degradation products |
| Specification | content | NMT 0.5% |
| Initial | 101.8 | <LOQ |
| 4 weeks | 101.9 | <LOQ |
| 8 weeks | 103.1 | <LOQ |
| 13 weeks | 104.5 | <LOQ |
| 26 weeks | 107.1 | <LOQ |

The stability data demonstrated that there are no significant chemical or physical changes in the product over the 108 week period when stored at 25° C./60% RH or the 26 week period when stored at 40° C./75% RH.

TABLE 11
stability data of 40 mg dosage formulations stored at
25° C./60% RH (relative humidity) (% LC—% of
label content; NMT—not more than; LOQ—level of quantification)

| Duration | Ketamine Content (% LC) | |
|---|---|---|
| Stability | 90.0-110.0% of nominal | Total degradation products |
| Specification | content | NMT 0.5% |
| Initial | 100.5 | <LOQ |
| 4 weeks | 99.3 | <LOQ |
| 8 weeks | 98.1 | <LOQ |
| 13 weeks | 98.3 | <LOQ |
| 26 weeks | 101.4 | <LOQ |
| 52 weeks | 97.8 | <LOQ |
| 108 weeks | 95.0 | <LOQ |
| 156 weeks | 104.8 | <LOQ |

TABLE 12
stability data of 40 mg dosage formulations stored at
40° C./75% RH (relative humidity) (% LC—% of
label content; NMT—not more than; LOQ—level of quantification)

| Duration | Ketamine Content (% LC) | |
|---|---|---|
| Stability | 90.0-110.0% of nominal | Total degradation products |
| Specification | content | NMT 0.5% |
| Initial | 100.5 | <LOQ |
| 4 weeks | 100.0 | <LOQ |
| 8 weeks | 98.3 | <LOQ |
| 13 weeks | 97.8 | <LOQ |
| 26 weeks | 100.7 | <LOQ |

The stability data demonstrated that there are no significant chemical or physical changes in the product over the 156 week period when stored at 25° C./60% RH or the 26 week period when stored at 40° C./75% RH.

### Dissolution Tests

Capsules from the stability studies also underwent dissolution tests throughout the study period as described below.

### Dissolution Method

1 L of the pH 1.2 buffer preparation was prepared by dissolving 2 g of sodium chloride and 7 mL of Hydrochloric acid in 1 L of analytical grade water.

A capsule was dissolved in 500 mL of degassed dissolution media (prepared as outlined above). The paddles were set to stir at 75 rpm and the dissolution media was at a temperature of approximately 37° C. (e.g. 37° C.±0.5° C.). Samples were withdrawn at the following time points: 0 minutes, 5 minutes, 10 minutes, 15 minutes and 30 minutes. The samples were analysed by HPLC and compared to a standard solution of ketamine hydrochloride.

The results from the HPLC analysis of the dissolution samples are detailed in Tables 13 to 16. The capsules were analysed in sets of six for each of the formulations, with the average of the six results reported.

TABLE 13
results of dissolution tests of 10 mg dosage formulations
stored at 25° C./60% RH (relative humidity)

| Duration | Dissolution |
|---|---|
| Stability Specification | Not less than 75% in 30 minutes |
| Initial | Mean 106% (Range 103% to 108%) |
| 4 weeks | Mean 106% (Range 102% to 110%) |
| 8 weeks | Mean 107% (Range 104% to 111%) |
| 13 weeks | Mean 106% (Range 105% to 107%) |
| 26 weeks | Mean 106% (Range 97% to 111%) |
| 52 weeks | Mean 105% (Range 102% to 109%) |
| 108 weeks | Mean 104% (Range 99% to 107%) |

TABLE 14
results of dissolution tests of 10 mg dosage formulations
stored at 0° C./75% RH (relative humidity)

| Duration | Dissolution |
|---|---|
| Stability Specification | Not less than 75% in 30 minutes |
| Initial | Mean 106% (Range 103% to 108%) |
| 4 weeks | Mean 104% (Range 102% to 110%) |
| 8 weeks | Mean 106% (Range 100% to 108%) |
| 13 weeks | Mean 107% (Range 103% to 111%) |
| 26 weeks | Mean 105% (Range 102% to 109%) |

TABLE 15
results of dissolution tests of 40 mg dosage formulations
stored at 25° C./60% RH (relative humidity)

| Duration | Dissolution |
|---|---|
| Stability Specification | Not less than 75% in 30 minutes |
| Initial | Mean 103% (Range 101% to 105%) |

| | | |
|---|---|---|
| 4 | weeks | Mean 102% (Range 101% to 103%) |
| 8 | weeks | Mean 104% (Range 101% to 110%) |
| 13 | weeks | Mean 103% (Range 100% to 103%) |
| 26 | weeks | Mean 103% (Range 103% to 105%) |
| 52 | weeks | Mean 102% (Range 100% to 104%) |
| 108 | weeks | Mean 102% (Range 100% to 104%) |
| 156 | weeks | Mean 105% (Range 100% to 105%) |

TABLE 16
results of dissolution tests of 40 mg dosage formulations
stored at 40° C./75% RH (relative humidity)

| Duration | Dissolution |
|---|---|
| Stability Specification | Not less than 75% in 30 minutes |
| Initial | Mean 103% (Range 101% to 105%) |
| 4 weeks | Mean 101% (Range 100% to 102%) |
| 8 weeks | Mean 103% (Range 101% to 106%) |
| 13 weeks | Mean 102% (Range 101% to 104%) |
| 26 weeks | Mean 101% (Range 101% to 104%) |

All formulations reached the criteria for immediate release, with at least 75% release being observed in 30 minutes. This was maintained throughout the duration of each stability study.

### INITIAL CLINICAL EXAMPLES

### Example 1

A patient already diagnosed with major depression disorder has failed on existing anti-depressant treatments (excluding ketamine) and is prescribed standard dose IV, subcutaneous, sublingual or intramusular ketamine treatment in a clinic under supervision.

On day 1 intravenous (IV) ketamine is administered. On day 2, 3, 4, 5, 6 or a combination thereof the patient is prescribed an oral ketamine composition as described herein which they take at home prior to their next supervised dose of IV, subcutaneous, sublingual, or intramusular ketamine.

The treatment can be supplemented with psychotherapy.

### Example 2

A patient already diagnosed with major depression disorder has failed on existing anti-depressant treatments (including ketamine treatments, but excluding oral ketamine hydrochloride capsules/compositions described herein) and is prescribed standard dose IV, subcutaneous, sublingual or intramuscular ketamine treatment in a clinic under supervision.

On Day 1 IV ketamine is administered. On day 2, 3, 4, 5, 6 or a combination thereof the patient is prescribed an oral ketamine composition as described herein which they take at home prior to their next supervised dose of IV, subcutaneous, sublingual or intramuscular ketamine.

The treatment can be supplemented with psychotherapy.

As used in examples 1 and 2, patients who have “failed on” existing treatments whether ketamine-based or not, or whether considered “treatment-resistant” or not, have not shown meaningful improvements on depression, mood or mental health rating scales and in particular in quality of life scales with those existing medications or treatments already tested.

Following an oral ketamine treatment regimen as described in examples 1 and 2, responding patients can expect meaningful improvements as assessed by rating scales and quality of life assessments in the order of 5% to 100%, with 100% representing full remission from depression, at least for a period of time, with some patients experiencing long periods of remission of up to 6 months or more.

After initial ketamine treatment (by IV, subcutaneous, intramuscular, intranasal or sublingual routes), the dosage of oral ketamine hydrochloride capsules/compositions is reduced so that oral ketamine hydrochloride capsules/compositions become the primary maintenance therapy for patients to be administered twice or three times weekly.

### Example 3

A patient suffering from acute suicidality is administered an oral ketamine composition as described herein at a minimum dosage of 40 mg daily for up to 3 weeks.

The treatment is continued until the symptoms associated with acute suicidality subside and/or other support services can be organised around the patient.

### Example 4

Patients diagnosed with Treatment Resistant Depression (TRD) receive a combined ketamine subcutaneous-oral treatment.

The patients are administered oral capsules comprising ketamine of dose range 120-160 mg once a week, four days after ketamine subcutaneous (0.5 mg-1.0 mg/kg) has been administered once a week.

The patients receive the combined ketamine subcutaneous-oral treatment for a period of between 1 and 12 weeks.

The mean MADRS reduction was recorded for each patient between the first and last treatment on subcutaneous-oral ketamine treatment.

### Example 5

Six patients diagnosed with Treatment Resistant Depression (TRD) received a combined ketamine treatment as outlined below. Their mean age was 53.1 (SD: 9.3). Five of the patients had previously attempted suicide and been admitted to a psychiatric unit. Three patients had received electroconvulsive therapy (ECT).

Five of the patients received oral ketamine of dose range 120-240 mg, (for example three to six capsules of 40 mg/day as described herein), once a week, four days after ketamine subcutaneous (0.5 mg-1.0 mg/kg) was administered once a week.

One patient received ketamine intravenously once a week and oral ketamine twice a week of dose 240 mg (for example, six capsules of 40 mg/day as described herein) each time.

The patients received on average 30.8 weeks (SD:7.7) ketamine subcutaneous-oral treatment and 10.6 weeks (SD: 6.7) ketamine IV-oral treatment.

The mean MADRS reduction was −5.7 (SD: 11.0) between the first and last treatment on subcutaneous-oral ketamine treatment and −5.7 (SD: 10.0) on intravenous-oral ketamine treatment. The patient that has received ketamine IV once a week and oral ketamine twice a week 240 mg reported significant improvement of cognition that lasts 24-36 h. The treatment regimens showed good response and tolerability in all patients after 45 to 75 minutes after dosing.

Treatment with oral ketamine was well-tolerated. No serious side effects occurred, and none of the participants discontinued treatment prematurely. No participant reported ketamine cravings or an urge to use ketamine beyond the prescribed treatment period. Onset of moderate discomforts or moderate increase of pre-existing discomforts was reported 32 times and mostly involved cognitive dysfunction (n=5), difficulty finding words (n=4), and nausea/vomiting (n=4).

Most of the participants reported positive effects, including reduction of suicidal thoughts, improved mood, cognition, and increased energy.

This pilot study was conducted to explore the safety, tolerability, and potential clinical effectiveness of a treatment with repeated, low-dose oral ketamine capsules. Most adverse events were moderate and self-limiting.

All patients had severe and chronic TRD and high levels of treatment refractoriness, including for ECT. Therefore, any clinical benefit in this patient category is a major gain. These results support the hypothesis that oral ketamine (such as the oral capsules described herein) is a safe and well-tolerated treatment for patients with chronic and severe TRD, in particular, when used in a combined treatment regimen with ketamine subcutaneous and/or intravenous, even after unsuccessful ECT.

### Example 6

Patients (n=12 patients) were administered a ketamine-based treatment comprising a subcut/IV ketamine dose with a supplemental oral dose (as described herein).

The results of this study show that the patients demonstrated remission via MADRS scores in the region of 50%.

Indeed the response was so good that the patients could be moved to an oral-only based ketamine treatment. When switched to an oral only treatment, the patients maintained their remission levels at the same level as when administered the combination treatment.

The patients did not reduce beyond the baseline level established with the full combination of Subcut/IV+Oral when moving to Oral alone.

This reduced hospital visits from once a week for Subcut/IV supervised treatment to once a month.

In terms of oral-based ketamine treatments, patients are started in the range of 120 mg-240 mg per day (often administered as multiple smaller doses up to the maximum required daily dose). In some cases they are started on 160 mg per day. Putative therapeutic ketamine ranges may be in the region of 160 mg-240 mg per day. Some patients are on 200 mg-240 mg per day as a standard treatment administered once or twice/week. Oral treatment twice a week is the most standard maintenance dose and seems to provide a considerable benefit.

An exemplar treatment schedule may be:
1: ketamine administered via IV/Sub cut
2: supplemented with oral ketamine
3: supplemented with oral ketamine
4: move patient to oral only treatment

1. ketamine or a pharmaceutically acceptable salt thereof in combination with a second anti-depressant agent, for use in treating, preventing controlling and/or managing a depressive disorder or a condition selected from pain, a neurological disorder, a psychological disorder, a mental health disorder, treatment resistant depression or a psychiatric disorder.

2. The ketamine for use of claim 1, wherein the ketamine is an enantiomer of ketamine, a ketamine derivative, a ketamine metabolite or a pharmaceutically acceptable salt thereof

3. The ketamine for use of claims 1-2, wherein the ketamine is administered orally.

4. The ketamine for use of claim 3 or method of claim 3, wherein the ketamine is administered orally once a week.

5. The ketamine for use of claims 3-4, wherein the ketamine is administered orally at a dose of 120 mg-240 mg per day.

6. The ketamine for use of claims 3-5, wherein the ketamine is administered orally at 40 mg 3 times per day.

7. The ketamine for use of claim 3-6, wherein the ketamine is administered orally at the required dose for 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18, weeks, 19 weeks, 20 weeks, 21 weeks, 22 weeks, 23 weeks, 24 weeks, 25 weeks, 26 weeks, 27 weeks, 28 weeks, 29 weeks or 30 weeks.

8. The ketamine for use of any preceding claim, wherein the second anti-depressant agent is selected from the group consisting of selective serotonin reuptake inhibitors (SSRIs), serotonin-noradrenaline reuptake inhibitors (SNRIs), noradrenaline and specific serotonergic antidepressants (NASSAs), tricyclic antidepressants (TCAs), monoamine oxidase inhibitors (MAOIs), and other ketamine formulations (e.g. injectable ketamine).

9. The ketamine for use of any preceding claim, wherein the second anti-depressant agent is administered subcutaneously or intravenously, optionally wherein the second anti-depressant agent is ketamine or a pharmaceutically acceptable salt thereof.

10. The ketamine for use of any preceding claim, wherein the second anti-depressant agent is administered subcutaneously at a dose of 0.5 mg-1.0 mg/kg.

11. The ketamine for use of any preceding claim, wherein the second anti-depressant agent is administered subcutaneously for 1 week, 2 weeks, 3 weeks, 4 weeks, 5 weeks, 6 weeks, 7, weeks, 8 weeks, 9 weeks, 10 weeks, 15 weeks, 20 weeks, 25 weeks, 30 weeks, 35 weeks or 40 weeks.

12. A subcutaneous or IV dose of ketamine and an oral dose of ketamine for use in treating, preventing, controlling treatment-resistant depression.

13. The subcutaneous/IV and oral ketamine dose for use of claim 12, wherein the ketamine is an enantiomer of ketamine, a ketamine derivative, a ketamine metabolite or a pharmaceutically acceptable salt thereof.

14. The subcutaneous/IV and oral ketamine dose for use of claims 12-13, wherein the subcutaneously/IV ketamine is administered at a dose of 0.5 mg-1.0 mg/kg once a week for three to four weeks.

15. The subcutaneous/IV and oral ketamine dose for use of claims 12-14, wherein the ketamine is administered orally at a dose of 120-240 mg per day, once a week for 1 to 30 weeks.

16. The subcutaneous/IV and oral ketamine dose for use of claims 12-15, wherein the ketamine is administered orally at a dose of 120-240 mg per day, once a week for between 1 and 8 weeks and then twice a week thereafter.

17. Subcutaneous or intravenous ketamine and oral ketamine for use in bringing a depressive disorder, including TRD, into remission.

18. Use of oral ketamine to maintain a depressive disorder, including TRD, in remission, wherein subcutaneous or intravenous ketamine combined with an oral ketamine dose is first used to bring a depressive disorder into remission and then an oral dose of ketamine is used to maintain the depressive disorder, including TRD, in remission.

19. A method of bringing a depressive disorder, including TRD, into remission, said method comprising administering a subject with a depressive disorder, an effective amount or remission inducing amount, of subcutaneous or intravenous ketamine and an effective amount or remission inducing amount of oral ketamine.

20. A method of maintaining a depressive disorder, including TRD, in remission, comprising subjecting a subject to the method of claim 19 to bring a depressive disorder, including TRD, into remission and then administering the subject an oral dose of ketamine in order to maintain the depressive disorder, including TRD, in remission.

21. A composition comprising:
(i) an active pharmaceutical agent, wherein the active pharmaceutical agent is selected from the group consisting of ketamine, enantiomers of ketamine, ketamine derivatives and ketamine metabolites, or a pharmaceutically acceptable salt thereof; and
(ii) one or more excipients selected from the group consisting of diluents, lubricants and compression aids.

22. A composition according to claim 21, wherein the composition is formulated for oral administration.

23. A composition according to claim 21 or 22, wherein the composition is provided in an encapsulated form, optionally wherein the composition is provided within a capsule.

24. A composition according to any one of claims 21-23, wherein the active pharmaceutical agent is selected from the group consisting of ketamine, ketamine hydrochloride, norketamine and hydroxynorketamine.

25. A composition according to any one of claims 21-24, wherein the active pharmaceutical agent is present in an amount between about 1 wt % and 50 wt % by total weight of the composition, or between about 5 wt % and 30 wt %, or between about 10 wt % and 25 wt % of the active agent by total weight of the composition.

26. A composition according to any one of claims 21-25, wherein the one or more excipients comprise lactose, optionally wherein the lactose is present in the composition in an amount between about 55 wt % to 99 wt %, or between about 65 wt % to 90 wt % by total weight of the composition.

27. A composition according to any one of claims 21-26, wherein the one or more excipients comprise:
(i) stearic acid or a metal stearate, such as magnesium stearate; and/or
(ii) a cellulose derivative, such as microcrystalline cellulose.

28. A composition according to any one of claims 21-27, wherein the composition comprises:
(i) ketamine hydrochloride
(ii) lactose monohydrate;
(iii) magnesium stearate; and
(iv) optionally microcrystalline cellulose.

29. A composition according to any one of claims 21-28, wherein the Compressibility Index of the composition is 25 or less.

30. A composition according to any one of claims 23-31, wherein the active pharmaceutical agent maintains at least 95% of an original level of pharmaceutical activity when stored within the composition for a period of at least 12 months at about 25° C. and 60% relative humidity.

31. A method of making a composition according to any one of claims 21 to 30, wherein the method comprises:
contacting, combining, mixing and/or blending one or more excipients together with the active pharmaceutical agent to provide the composition.

32. A method of making a composition according to claim 31, comprising:
(a) mixing and/or blending lactose and optionally a cellulose derivative with the active pharmaceutical agent to provide a pre-mixed and/or pre-blended composition; and
(b) mixing and/or blending a metal stearate with the pre-mixed and/or pre-blended composition to provide the composition.

33. A method according to claim 31 or 32, further comprising:
filling a capsule with the composition.

34. A composition according to any one of claims 21 to 30 for use as a medicament.

35. A composition according to any one of claims 21 to 30 for use in the treatment, prevention, control and/or management of a condition selected from pain, a neurological disorder, a psychological disorder, a mental health disorder or a psychiatric disorder.

36. A composition for use according to claim 35, wherein the condition is a depressive disorder or treatment-resistant depression.

37. A composition for use according to claims 35 to 36, wherein the composition is administered to a subject in need thereof to treat, prevent, control and/or manage suicidal ideation or behaviour associated with a mental health disorder or psychiatric disorder.

38. A composition for use according to any one of claims 35 to 37, wherein the composition is orally administered to a subject in need thereof.

39. An active pharmaceutical agent selected from the group consisting of ketamine, enantiomers of ketamine, ketamine derivatives and ketamine metabolites, or a pharmaceutically acceptable salt thereof, for use in treating a depressive disorder in a subject in need thereof, wherein the active pharmaceutical agent is:
(i) administered orally; and
(ii) administered in combination with a second anti-depressant agent.

40. An active pharmaceutical agent for use according to claim 39, wherein the second anti-depressant agent is selected from the group consisting of selective serotonin reuptake inhibitors (SSRIs), serotonin-noradrenaline reuptake inhibitors (SNRIs), noradrenaline and specific serotonergic antidepressants (NASSAs), tricyclic antidepressants (TCAs), monoamine oxidase inhibitors (MAOIs), and other ketamine formulations (e.g. injectable ketamine).

41. An active pharmaceutical agent for use according to claim 39 or 40, wherein the active pharmaceutical agent is comprised within the composition of any one of claims 1 to 10.

42. An active pharmaceutical agent for use according to claim 41, wherein the active pharmaceutical agent is administered to a subject suffering from a depressive disorder who is beginning or undergoing treatment with the second anti-depressant agent.

43. An active pharmaceutical agent for use according to any one of claims 39 to 42, wherein the active pharmaceutical agent is administered to treat, control and/or prevent an instability in depression levels, a relapse in depression, an acute depressive episode and/or suicidal ideation and/or behaviour associated with a depressive disorder.

44. An active pharmaceutical agent for use according to any one of claims 39 to 43, wherein the active pharmaceutical agent:
(i) is administered during an initial period of a treatment regimen based on the use of the second anti-depressant agent; and/or
(iii) is administered during a period of instability, relapse, and/or acute depressive episode and/or a period where the subject is suffering from suicidal ideation and/or behaviour associated with a depressive disorder.

45. An active pharmaceutical agent for use according to any one of claims 39 to 44, wherein the active pharmaceutical agent is administered multiple times over a period of at least 1, 2, 3, 4, 5 or 6 weeks or up to 10, 11, or 12 weeks.

46. An active pharmaceutical agent for use according to any one of claims 39 to 45, wherein the second anti-depressant agent is ketamine that is administered by a different route, optionally wherein the second anti-depressant agent is ketamine that is administered subcutaneously, intravenously or intramuscularly.

47. A composition for use according to any one of claims 34 to 38, or an active pharmaceutical agent for use according to any one of claims 39 to 46, wherein the depressive disorder is selected from the group consisting of major depressive disorder, treatment resistant depression, dysthymia, post-partum depression, atypical depression (AD), melancholic depression, psychotic major depression, catatonic depression, double depression, depressive personality disorder, depressive disorder not otherwise specified, premenstrual dysphoric disorder, seasonal affective disorder (SAD), recurrent brief depression and minor depressive disorder.

48. A composition for use according to any one of claims 34 to 38, or an active pharmaceutical agent for use according to any one of claims 39 to 46, wherein the depressive disorder is treatment resistant depression.

49. IV/subcutaneous ketamine and oral ketamine for use in treating or preventing a depressive disorder and/or for use in bringing repressive disorder into remission and maintaining the state of remission, wherein a subject having a depressive disorder is administered IV/subcutaneous ketamine and oral ketamine and then only oral ketamine.

50. The IV/subcutaneous ketamine and oral ketamine for use of claim 49, wherein the ketamine is racemic ketamine, an enantiomer of ketamine, a ketamine derivative, a ketamine metabolite or a pharmaceutically acceptable salt thereof.

51. The IV/subcutaneous ketamine and oral ketamine for use of claim 49 or 50, wherein the IV/subcutaneous ketamine is administered at a dose of 0.5 mg/kg and 1.0 mg/kg.

52. The IV/subcutaneous ketamine and oral ketamine for use of claims 49-51, wherein the oral ketamine is administered at a dose of 120-240 mg per day.

53. The IV/subcutaneous ketamine and oral ketamine for use of claims 49-52, wherein the oral ketamine is administered for a longer period than the IV/subcutaneous ketamine.
